(19) United States
(12) Patent Application Publication
(10) Pub. No.: US 2009/0320837 A1
(43) Pub. Date: Dec. 31, 2009

(54) DRY POWDER INHALERS

(75) Inventors: Simon James Smith, Hertford (GB); David Stuart Harris, Cambridge (GB); Rachel Victoria Striebig, London (GB); Julie Suzanne Pearl, Sandy (GB)

Correspondence Address: CANTOR COLBURN, LLP, 20 Church Street, 22nd Floor, Hartford, CT, 06103, US

(73) Assignee: CAMBRIDGE CONSULTANTS LIMITED, Cambridge (GB)

(21) Appl. No.: 12/304,909

(22) PCT Filed: Jun. 13, 2007
(86) PCT No.: PCT/GB2007/002190

§ 371 Date: May 28, 2009

(30) Foreign Application Priority Data
Jun. 13, 2006 (GB) 0611659.4

(51) Int. Cl. A61M 15/00

(52) U.S. Cl. 128/203.15

(57) ABSTRACT

A dry powder inhaler comprises: a cyclone chamber having a cyclone chamber outlet (20) extending substantially axially therefrom; a mouthpiece channel (26) having a central axis; and a plenum portion (58) connecting said cyclone chamber outlet (20) and said mouthpiece channel, the mouthpiece channel axis Aₘ being offset from, and making a non-parallel angle with, the axis of the cyclone outlet A_{c} such that in use air exits the cyclone outlet (20) at least partly tangentially into the mouthpiece (28). Also disclosed is a blister pack (200) for use with a dry powder inhaler and including a dose of powder for inhalation, the blister comprising: a base part (202) comprising at least one chamber (204,206); a grommet cover (208) extending partly over said chamber and defining an aperture (212, 214) above the chamber; and a protective layer (210) covering at least said aperture, wherein the edge of the grommet cover (208) defining the aperture (212, 214) is compliant so as in use to form a seal around a tube passing through the aperture.

This invention relates to inhalers for delivering substances in powder form to the respiratory system of a user by inhalation; and to receptacles for such powder.

Dry Powder Inhalers (DPIs) are conventionally used to deliver active drug substances to the lungs of a user to treat asthma and other respiratory diseases. The basic principle upon which such inhalers work is that the user holds the inhaler to his or her mouth and draws breath through the device, thereby setting up a flow of air which entrains drug particles so that they are drawn into the user's respiratory system. The drug may be in the form of a free powder, or more commonly the drug is bound to carrier particles such as lactose. Of course, a blend of drug particles may be used.

The combined, aggregate particle size of the drug particle and carrier particle is generally greater than 1-5 μm (microns) which is the target size range for particles to be effectively inspired into the deep part of the lungs. DPIs therefore need to de-aggregate the particles (that is to separate the drug particles of respirable size from the larger carrier particles).

Furthermore, there is a tendency for the respirable particles to aggregate during storage. The DPI should therefore de-aggregate these fine (respirable) particles. Despite this, known DPIs are rather inefficient at de-aggregating the drug particles. The number of particles of respirable size as a proportion of the total output of the inhaler is known as the Fine Particle Fraction (FPF) . In typical conventional inhalers the Fine Particle Fraction can be as low as 30%, and 40-50% is typical. Moreover, in many devices the FPF is dependent upon the inhalation flow rate of the user so that performance is inconsistent both between users and from one use to the next. Of course, a low FPF also leads to much of the drug being wasted. The additional problem with the FPF being inconsistent is that it is then impossible to control the dose actually being received by the user.

A low FPF is of particular concern since some of the particles which are not fully inhaled tend to hit the back of the user's throat and are deposited there. There is some evidence to suggest a link between deposition of steroid-based drugs on a user's throat and an increased risk of throat or lung cancer. A further problem with existing dry powder inhalers is that the carrier particles (e.g. lactose) also tend to be inhaled and hit the back of the throat which gives rise to an unpleasant gritty feel. The build up of lactose also can be a contributing factor towards thrush.

Existing designs also suffer from the problem of particles being deposited on the walls of the inhaler itself. Whilst deposition on the apparatus is preferable to deposition on the back of the throat, it can give rise to a further problem when the deposited powder is dislodged in a subsequent use since this will adversely affect the uniformity of dose received by a user.

It is an object of the invention to provide a dry powder inhaler which alleviates at least some of the problems set out above.

When viewed from a first aspect the invention provides a dry powder inhaler comprising: a cyclone chamber having a cyclone chamber outlet extending substantially axially therefrom; a mouthpiece channel having a central axis; and a plenum portion connecting said cyclone chamber outlet and said mouthpiece channel, the mouthpiece channel axis being offset from, and making a non-parallel angle with, the axis of the cyclone outlet such that in use air exits the cyclone outlet at least partly tangentially into the mouthpiece.

In accordance with the invention the swirling air exiting the cyclone chamber passes into the mouthpiece channel at least partly tangentially. This means that there is an increased tendency for the air flow to continue approximately linearly, generally parallel to the axis of the mouthpiece channel, rather than to continue swirling. this reduces the tendency for the entrained powder to be deposited on the inside of the mouthpiece. Arrangements in accordance with the invention have also been found to exhibit a lower inhalation resistance which allows a smaller mouthpiece to be employed without adversely increasing the work of inhalation required of the user. Accordingly the overall device can be made more compact.

The cyclone chamber may be an integral part of the inhaler or could be provided partly or fully by a replaceable part so that it can be disposable. Accordingly the invention extends to an inhaler without a cyclone chamber actually present. Thus when viewed from a second aspect the invention provides a mouthpiece arrangement for a dry powder inhaler comprising a cyclone chamber outlet for connection in use with a cyclone chamber; a mouthpiece channel having a central axis; and a plenum portion connecting said cyclone chamber outlet and said mouthpiece channel, the mouthpiece channel axis being offset from, and making a non-parallel angle with, the axis of the cyclone chamber outlet.

The axis of the mouthpiece channel could extend from the plenum portion perpendicularly or even at an acute angle, but in preferred embodiments the mouthpiece channel extends at an obtuse angle from the cyclone outlet axis since this allows it to match more closely the trajectory of helically flowing air exiting the cyclone chamber, and thereby minimise deposition and flow resistance.

The mouthpiece channel could be tapering, parallel-sided, or flared as convenient.

The cyclone chamber may be of any configuration which gives rise to a helically circulating flow of air, but in accordance with at least some preferred embodiments of the invention the cyclone chamber is so shaped that at least a part of the chamber decreases in cross-sectional area in a direction away from its air inlet, so as thereby in use to set up a reverse flow cyclone in the chamber.

Thus in such embodiments the airflow path includes a reverse-cyclone chamber. The required powdered substance is entrained in the air which passes through the cyclone chamber in which a reverse-flow cyclone is set up. The reverse flow cyclone referred to herein has a particular meaning distinct from the general usage of the term cyclone in the art to mean any form of circulating air. A reverse-flow cyclone is one in which the air circulates in two generally concentric columns in opposite axial directions.

This arrangement is particularly advantageous in the present application for a number of reasons.

Firstly, the flow pattern in a reverse-flow cyclone - with an outer, downwardly spiraling “free” vortex and an inner, upwardly spiraling “forced” vortex—gives rise to a substantial fluctuation in tangential velocity across the width of the chamber. The steep velocity gradient encountered in the flow cause efficient de-aggregation of the particles. Moreover, the particles are subjected to these relatively high shear forces both as they travel downwardly to the base of the chamber and also as they travel back up the chamber in the inner, forced vortex. This relatively long flow path over substantially the whole of which de-aggregation can take place leads to a significantly increased proportion of fine particles within the entrained airflow as it travels towards the exit of the cyclone chamber. Furthermore the outer free vortex acts to scour the walls of the chamber of any fine powder particles deposited thereon.

Secondly, the central, forced vortex, which travels up from the base of the chamber is relatively tight and well defined. As is known in the art, the mean radius of circulation of a particle is dependent upon its weight and therefore size. Thus by careful selection of a particular circulation radius, a very sharp cut-off threshold of particle sizes may be achieved. By selecting a radius equivalent to 5 microns or less, an even higher Fine Particle Fraction may be achieved. Such selectivity can be obtained for example, by a “vortex finder” comprising a tube projecting some way into the cyclone chamber, which provides the outlet to the chamber. In preferred embodiments therefore the cyclone chamber outlet projects into the cyclone chamber to provide such a vortex finder.

Thirdly, the reversal of vertical direction of travel of the particles at the base of the chamber causes the de-aggregated carrier particles, and any drug or combination particles which are too large, to be trapped within the cyclone and thus not be inhaled by the user. This substantially reduces the deposition of large particles on the user's throat with the attendant problems referred to previously. The separation of the large particles retained in the inhaler from the finer particles which are inhaled is seen as an important benefit which may be achieved in accordance with these embodiments of the invention.

Fourthly, the residence time of the particles is greatly increased (therefore giving a greater number of opportunities for separation). Typically in a conventional DPI all drug is evacuated within 0.5 seconds. In accordance with preferred embodiments of the invention, particles remain within the device for the full duration of inhalation. This maximizes the shear forces for a given energy input.

As mentioned previously, in some embodiments the cyclone chamber could be provided as an integral part of the inhaler. A preferred example of such an embodiment would be one whereby the entire inhaler was disposable, Having the inhaler disposable would overcome any potential problem of powder deposited in the cyclone chamber affecting dose uniformity that might otherwise arise.

In other embodiments the cyclone chamber is provided fully or partially by a removable part to allow it to be disposable. In such embodiments the cyclone outlet could be attached to or integral with the cyclone chamber so as to be removable with it. The same applies to the plenum portion. This would further avoid problems arising from powder deposition on the apparatus walls.

In another set of embodiments it may be the mouthpiece which is removable for disposal so instead the plenum portion or cyclone chamber outlet may be attached to or integral with the mouthpiece for removal with it. Indeed it is conceivable that all four elements be disposable together; but that is little removed from having the whole inhaler disposable.

Where at least the cyclone chamber is provided on a replaceable part, it is preferred that one or more doses of the powdered substance is also provided on the replaceable part. This is a particularly advantageous arrangement since it simplifies the provision of the two “consumable” elements that is to say the powdered drug or other substance itself, and the chamber which is regularly replaced. In these embodiments the drug or the like could be metered from a reservoir in the replaceable part but it is preferred to provide one or more discrete doses. This simplifies construction which of course allows the production cost of the replaceable part to be minimized and, in accordance with another preferred feature, allows the doses to be individually sealed which protects them from contamination, especially by moisture and cross-contamination between used and unused doses.

A removable part for a dry powder inhaler comprising at least a cyclone chamber and a quantity of powdered substance for inhalation, is clearly an advantageous embodiment of the invention or included in advantageous embodiments. Where a plurality of discrete doses is provided these will of course often be identical to one another. However it is envisaged that in some embodiments it will be beneficial for the doses to vary in size.

One or a plurality of circulation or cyclone chambers may be provided on the replaceable part or, as mentioned above, they may be permanently mounted on or integral with the inhaler. In either case a plurality of doses could be associated with each chamber, i.e. so that a given chamber is reused a small number of times, but it is preferred that only a single dose is associated with the or each chamber. Also two or more drug powders could separately stored and mixed in a cyclone chamber e.g. with two or more tangential inlets to the cyclone chamber.

The replaceable part could be provided in general with one or a plurality of circulation or cyclone chambers and one or a plurality of powdered doses. Preferably the chamber and/or chambers is protected by a frangible membrane e.g. a polymeric or metallic foil to protect the formulation against environmental conditions.

Even where the cyclone chamber is not provided on a replaceable part, the drug or other powder could be. This would have the advantages mentioned above of isolation of the drug prior to use etc. In such arrangements, the drug is preferably released by the act of installing the replaceable part to the inhaler. For example, where the drug is stored in a frangible membrane, the inhaler could be arranged to pierce this when the replaceable part is installed.

The reverse-cyclone chamber of preferred embodiments including its air inlet will be arranged so that the necessary vortex is set up when a user inhales. Although there are other ways of achieving this, conveniently the air inlet is directed substantially tangentially. Preferably the air inlet is arranged to direct air in such a way that it circulates around the periphery of the chamber. Preferably the chamber has a cylindrical section in the region of the air inlet. This facilitates establishment of the free vortex airflow. Of course there could be more than one air inlet.

In general, the outlet from the chamber will be provided at approximately the same level as or below the air inlet. This maximizes the benefit given by the reverse-cyclone flow pattern.

In accordance with all embodiments of the invention, the dose of powder could be arranged to be introduced into and entrained by the inhaled air at any convenient point in the system. In one set of preferred embodiments the powder is stored within the cyclone chamber.

Alternatively, the powder could be entrained prior to entry into the cyclone chamber. For example this could take place in the conduit leading to the cyclone chamber,

Returning to the shape of the tapering area reverse-cyclone chamber, the Applicant has devised a some possible features whereby performance of the chamber may be enhanced. In some embodiments, the base of the cyclone chamber could generally conform to part of the surface of a toroid, which has been found in some circumstances to enhance the establishment of the reverse-cyclone flow pattern, but also more particularly to enhance tight local circulation of the larger particles which are trapped at the base of the cyclone chamber.

In another potential feature, the base of the cyclone chamber is provided with a series of concentric ridges i.e. it has a stepped profile. In some circumstances this can give a more desirable flow pattern.

In another potential feature vertical ridges may be provided in the chamber to enhance the performance.

In yet another possibility, the surface finish of the wall of the chamber could be made rough or smooth as desirable to give an appropriate flow pattern. The surface finish could even vary from rough to smooth or vice versa to influence the particular flow since the Applicant has observed that the roughness of the surface can affect the performance of the cyclone. Of course, any combination of the features mentioned above may be employed.

Where, as is preferred, the cyclone chamber is protected by a frangible membrane, this could be pierced upon installation into the inhaler e.g. when the dose is ready to be taken.

The dimensions of the inhaler may be chosen to suit the particular desired application. However the features set out herein are especially advantageous in inhalers which can be held in one hand. Preferably the diameter of the cyclone chamber is between 5 and 100 mm, more preferably between 5 and 50 mm and most preferably between 8 and 20 mm.

The air inhaled by a user may all be drawn through the cyclone chamber and plenum portion. However in accordance with preferred embodiments of the invention the inhaler comprises a main airflow path which passes through the cyclone chamber and a bypass airflow path bypassing the cyclone chamber; wherein the main and bypass airflow paths communicate with the mouthpiece.

In accordance with such embodiments, only a proportion of the air inhaled by a user is drawn through the cyclone chamber. The remainder is drawn through the bypass airflow path into the mouthpiece without passing through the cyclone chamber. The Applicant has found that even though in accordance with the invention the flow resistance exhibited by the inhaler tends to be reduced, in some circumstances bypass airflow is nonetheless important in limiting the flow rate through the cyclone chamber, and controlling the overall device airflow resistance as felt by the user. If there is too great a flow rate through the cyclone chamber, then the velocity of the particles is too great and so even the fine respirable particles are separated and hence retained in the cyclone. Therefore the cyclone must be sufficiently large to allow the respirable particles to escape for a given flow rate. In practice this could mean that the chamber would be too large to be incorporated in an easily portable device such as can be carried in a pocket or handbag.

However by using the bypass, the flow rate through the chamber may be limited without having to increase the overall inhalation resistance of the inhaler, which would undesirably increase the time required for a user to draw a full breath through the device.

The relative resistances of the main and bypass airflow paths may be set during manufacture so as to give a predetermined flow rate through cyclone at a standard average inhalation flow rate. This has been found to give good results. However, it is envisaged that it might be possible to increase even further the consistency of the Fine Particle Fraction and the delivered dose by providing means for varying the flow resistance of the bypass air flow path such that said resistance is decreased at increasing inhalation flow rates. In accordance with such a feature, the flow rate through the cyclone chamber may be kept more consistent even in the face of a varying rate of inhalation by the user since the resistance in the bypass path will automatically adjust with the user's rate of inhalation. For example, if the user inhales harder than average, the resistance in the bypass airflow path will decrease thereby allowing a greater bypass airflow to meet the excess flow rate without increasing the flow rate through the cyclone chamber to the same extent or, ideally, at all.

The above mentioned variable flow resistance in the bypass path could be achieved in a number of ways. In a simple example, one or more resiliently biased flaps could be provided extending across all or part of the bypass airflow path. In one convenient embodiment envisaged, a star-valve could be utilized. These generally comprise a plug of resilient material across a tube with a series of radial slits which allow individual segments to flex outwardly thereby allowing fluid to flow past the valve. The characteristics of such valves is that as the flow rate of fluid through them increases, the deflection of the individual segments also increases, thereby enlarging the generally star-shaped aperture which is created. Such a structure is commonly to be found on domestic containers for viscous fluids such as sauces, toiletries etc.

It is not critical to the invention where the bypass air flow and the main airflow meet. For example they could meet before the plenum portion, in the plenum portion or after it. For example the bypass air flow could simply be provided by an aperture in the mouthpiece. Preferably the main and bypass airflows do not meet at all inside the inhaler. This is achieved by providing a divided mouthpiece, with one channel for the main airflow and the other channel for the bypass airflow. Preferably the bypass airflow is provided via a grille in the body of the inhaler.

A further invention disclosed herein relates to blister packs for inhalers, typically containing medicament powder for inhalation by a user. This invention is suitable for use with the inhalers described above but may equally be used with other inhalers.

The previous invention has been discussed with reference to embodiments that employ disposable blister packs with a foil membrane cover. In particular blister packs comprising a reverse-cyclone chamber and powdered medicament have been discussed. The Applicant has found that in practice a foil membrane which is pierced by a tube on the inhaler may not provide sufficient sealing around the piercing tube to prevent air being drawn into the cyclone chamber around the outlet/piercer tube. It has also recognised that the flaps of foil around the edge of the pierced hole can have an adverse effect on the desired airflow patterns. which can affect the efficiency of the inhaler.

The Applicant has devised a novel blister pack which is intended at least partly to alleviate this problem. According to a further invention disclosed herein there is provided a blister pack for use with a dry powder inhaler and including a dose of powder for inhalation, the blister comprising: a base part comprising at least one chamber; a grommet cover extending partly over said chamber and defining an aperture above the chamber; and a protective layer covering at least said aperture, wherein the edge of the grommet cover defining the aperture is compliant so as in use to form a seal around a tube passing through the aperture.

Thus it will be seen that in accordance with this invention a blister pack is provided which can form a seal around a tube inserted into the chamber of the blister.

The invention extends to a dry powder inhaler including such a blister pack. The inhaler preferably comprises a tube which can be introduced into the chamber through the aperture in the grommet cover, the tube being slightly larger than the aperture so that the grommet cover forms a seal around the tube.

The grommet cover could comprise any suitable material. Preferably the grommet cover is non-metallic. Preferably the edge of the grommet cover defining the aperture is elastically compliant. It could for example comprise a plastics or synthetic rubber material.

The protective layer could be frangible, e.g. a foil membrane which is permanently attached to the pack so that it must be pierced as is well known in the art. Preferably however the layer or a portion thereof is removable. For example it may be attached by means of a non-permanent adhesive so as to allow it to be peeled off. By avoiding the use of a foil membrane, preferred embodiments avoid the aforementioned problems which can be caused by the flaps of foil released after piercing.

The chamber may be of any desired configuration depending on the requirements of the inhaler with which it is intended to be used. Preferably the chamber is so shaped that at least a part of the chamber decreases in cross-sectional area in a direction away from its air inlet, so as thereby in use to set up a reverse flow cyclone in the chamber.

Preferably the blister pack recited above comprises a second aperture defined in a grommet cover with a compliant edge. This allows e.g. sealing around an inlet and an outlet tube. The grommet cover could be the same as the first-recited one or it may be a separate, second grommet cover. The second aperture may be above the previously-recited chamber, a second chamber or an air conduit formed on the blister.

This invention is also applicable to closure of a chamber which is not on a blister pack but is part of an inhaler—e.g. by being formed integrally therewith. Thus when viewed from a further aspect the invention provides a dry powder inhaler comprising a dose of powder for inhalation, a body part defining at least one chamber; a grommet cover extending partly over said chamber and defining an aperture above the chamber; and a protective layer covering at least said aperture, wherein the edge of the grommet cover defining the aperture is compliant so as in use to form a seal around a tube passing through the aperture.

The inhaler preferably further comprises a tube which can be introduced into the chamber through the aperture in the grommet cover, the tube being slightly larger than the aperture so that the grommet cover forms a seal around the tube.

The preferred features of the blister pack apply equally to the arrangement whereby the chamber is part of an inhaler.

Certain preferred embodiment of the inventions will now be described, by way of example only, with reference to the accompanying drawings in which:

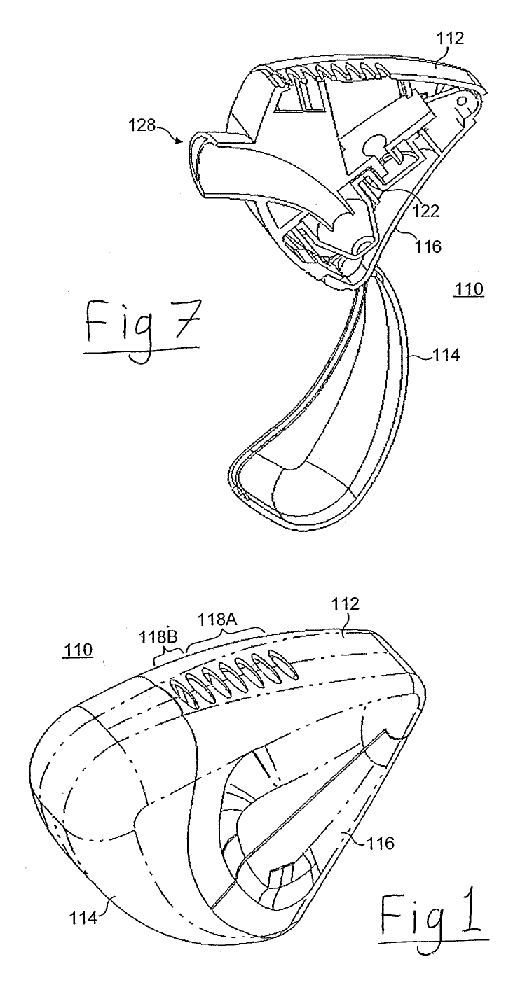

FIGS. 1 to 7 are schematic and sectional views of an inhaler described for the purposes of reference only;

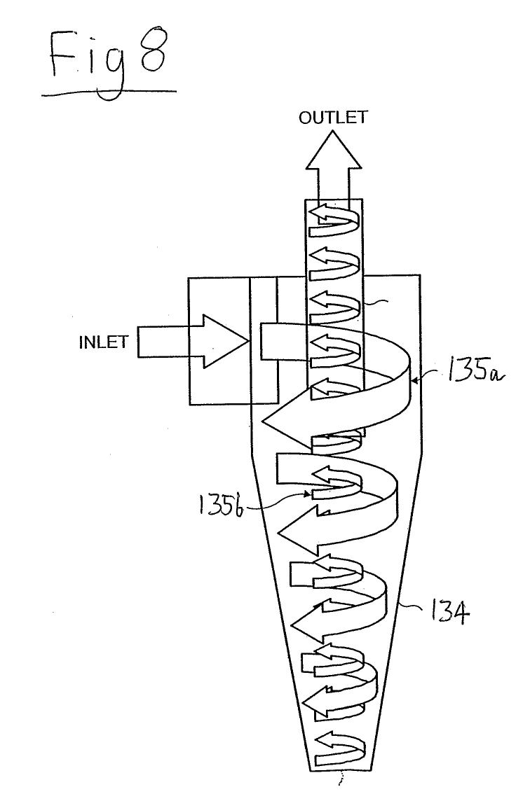

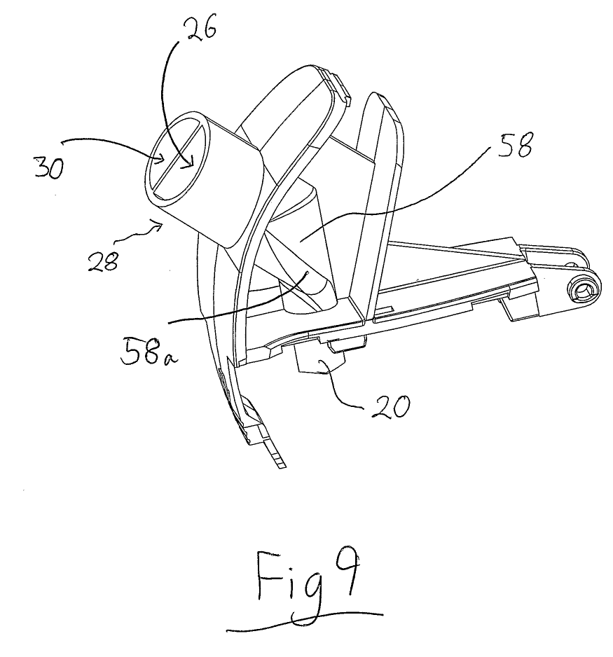

FIG. 8 is a schematic view of a reverse-cyclone flow in a frusto-conical chamber.

FIG. 9 is a perspective view of a modified piercer/mouthpiece structure in accordance with the invention;

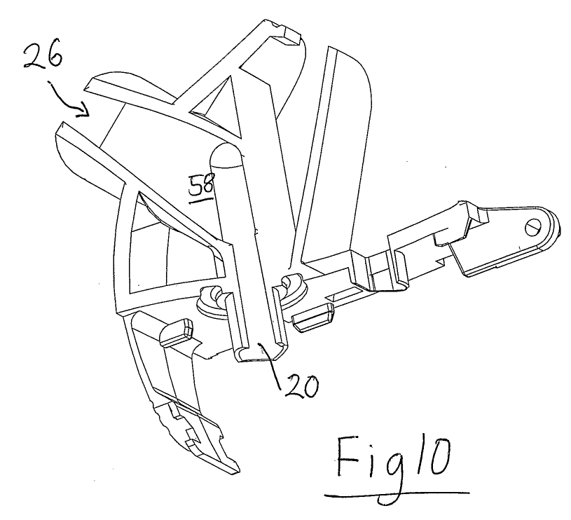

FIG. 10 is a cross-sectional view of the structure of FIG. 9;

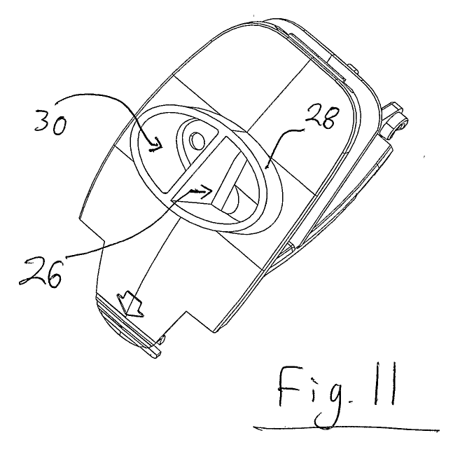

FIG. 11 is a view of the structure of FIG. 9 looking into the mouthpiece.

FIG. 12 is a schematic diagram illustrating the offset arrangement exhibited by the structure of FIGS. 9 to 11;

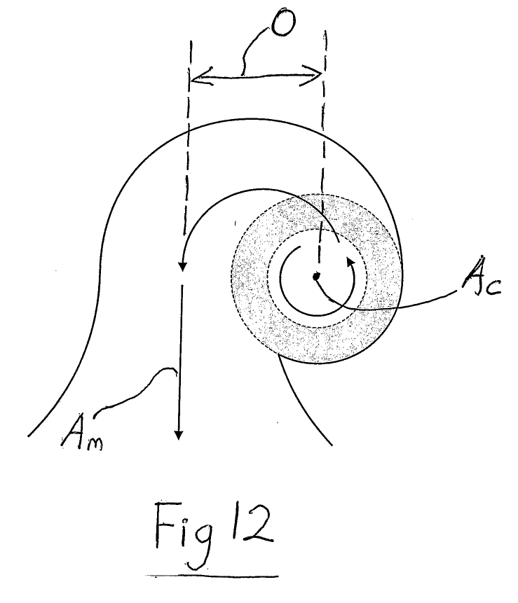

FIG. 13 shows five different cyclone chamber configurations A-E used to test the performance of a reverse flow cyclone;

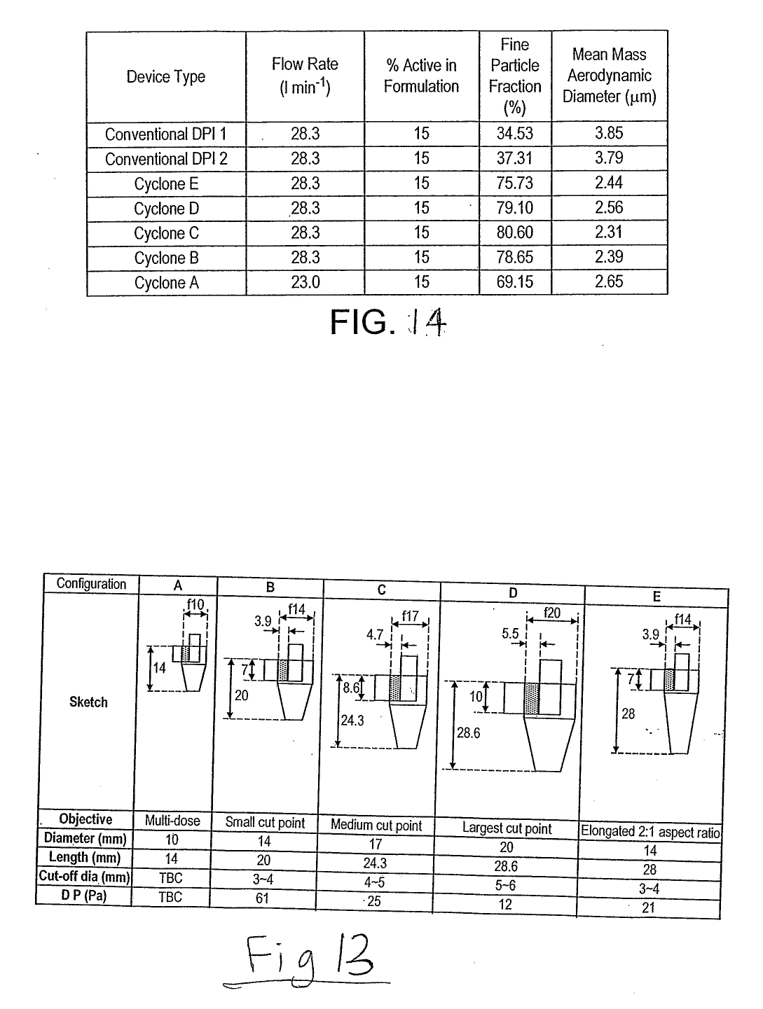

FIG. 14 shows the performance test results for the cyclone chambers A-E of FIG. 13 compared to two conventional dry powder inhalers;

FIG. 15 is a perspective view of a blister pack in accordance with a further invention disclosed herein;

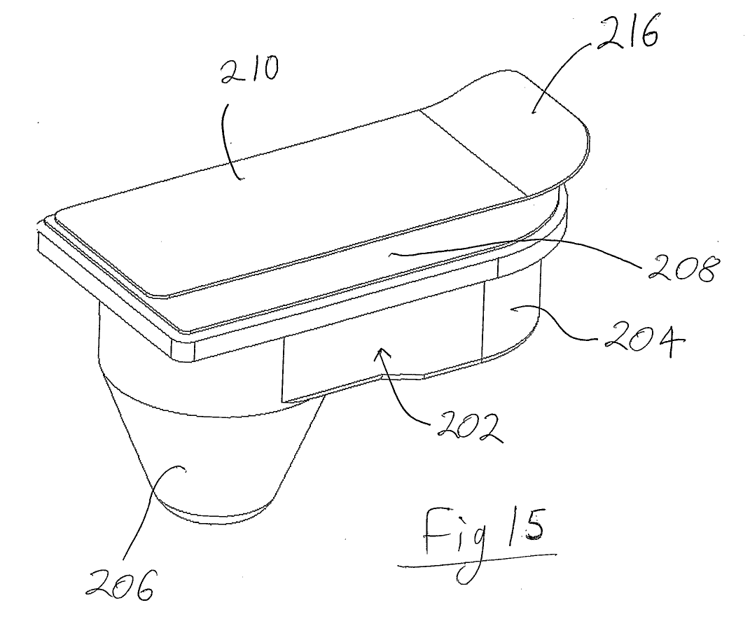

FIG. 16 is a perspective view similar to FIG. 15 but with the protective layer removed; and

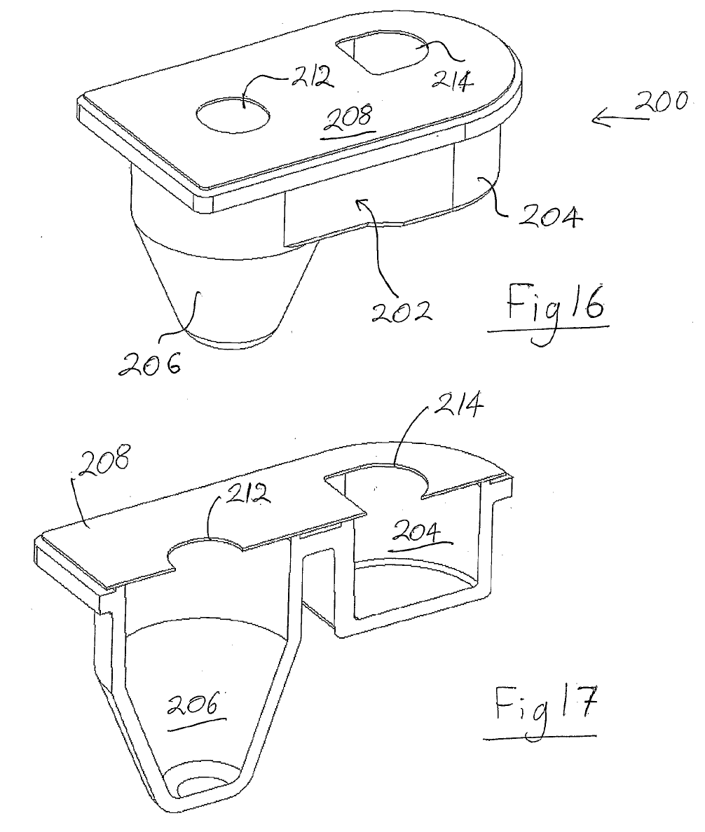

FIG. 17 is a sectional view of the blister pack of FIG. 15.

An inhaler is shown in FIGS. 1 to 7 and will be described for the purposes of reference only. The dry powder inhaler 110 is shown having an outer casing 112, a hinged mouthpiece protector 114, and a dose holder 116. The illustrated casing 112 includes seven air inflow ports, five denoted by reference numeral 118A and two denoted by reference numeral 118B, together forming two grilles. The inhaler 110 is adapted to dispense dry powder drug/medicament along a main airflow path (MP) extending from ports 118A, through a first piercing tube 124 (not visible in FIG. 1), a foil-faced dose container blister pack 122 (not visible in FIG. 1), a second piercing tube 120 (not visible in FIG. 1), and a drug port 126 in a mouthpiece 128 (not visible in FIG. 1) positioned beneath the mouthpiece protector 114. A secondary bypass airflow path (SP) within the casing 112 extends from ports 118B directly to a bypass port 130 (not visible in FIG. 1) in mouthpiece 128.

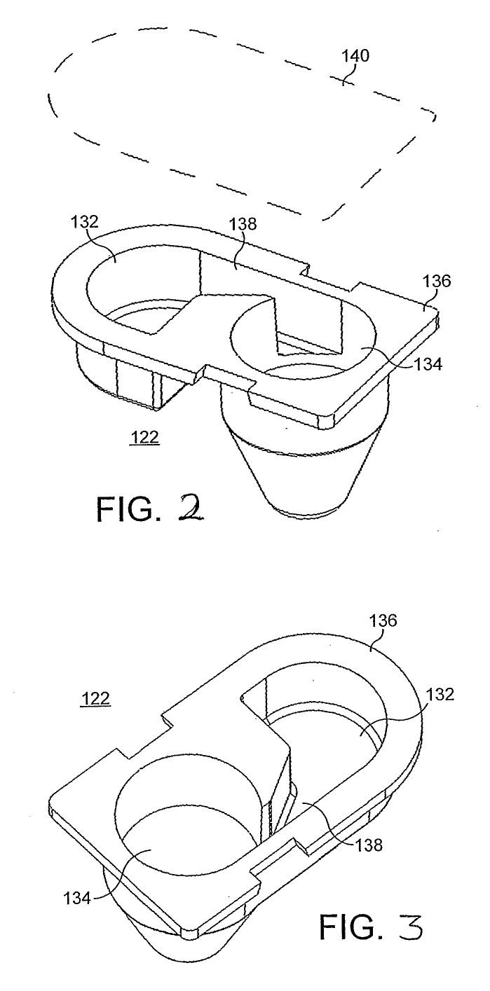

The dose container blister pack 122 is shown (without its foil facing) in FIGS. 2 and 3, although the foil-facing is shown in phantom in FIG. 2 displaced from and above the main portion of dose container 122. The dose container 122 is made from a moulded plastics material and includes an inlet chamber 132 and a cyclone chamber 134 extending from a planar face member 136. A channel 138 interconnects chambers 132 and 134. A pierceable foil-facing or laminate 140, shown in phantom in FIG. 2, and not shown in FIG. 3, is affixed to the face member 136 and spans and hermetically seals the chambers 132 and 134. The inlet chamber 132 serves as a reservoir for dry powder medicament-to-be-dispensed. The cyclone chamber 134 serves as a de-agglomerating airflow guide, adapted to effect a separation of relatively small medicament drug particles from relatively large carrier particles entrained in air flow along path MP in use. The chamber 134 is shaped to establish a reverse cyclone airflow, as will be described in greater detail below with reference to FIG. 11. The dose container blister pack 122 is shaped to removably interfit within the dose holder 116.

The casing 112 houses a piercer/mouthpiece structure 150, shown in FIG. 4. The structure 150 includes a base portion 152 from which an MP/SP channel divider 154 extends to an inner-surface of the casing 112, between the ports 118A and 118B. The structure 150 also includes a mouthpiece support member 156 extending therefrom, which supports the mouthpiece 128. A MP channel member 158 extends between the support member 156 and the base portion 152, and defines therein a portion of the main airflow path MP between the mouthpiece 128 and the first piercing tube 120, which extends downward (as shown in FIG. 4) from the base portion 152 into the cyclone chamber 134 to form a vortex finder. In the inhaler depicted in FIGS. 1 to 7, this main airflow path is a gentle continuous curve from the piercing tube/vortex finder 120 which forms the outlet of the cyclone chamber to the mouthpiece 128. Air that is circulating as it exits the cyclone chamber 134 is therefore likely to continue to circulate as it passes into the mouthpiece 128. This might lead to deposition of entrained particles onto the inner surface of the mouthpiece.

The second piercing tube 124 also extends downward (as shown in FIG. 4) from the base member 152. A pivot assembly 160 for pivotally supporting the dose holder 116 with respect to the support 150, extends from the leftmost (as shown in FIG. 4) portion of the base member 152. A collar portion 162 extends about the MP channel member 158 at the junction of MP channel member 158 and mouthpiece support 156. The collar portion includes a first SP port 164 on one side of MP channel member 158 and a second SP port 166 (not visible in FIG. 4) on the other side of channel member 158. The first and second SP ports 164 and 166 couple ports 118B and the region bounded by casing 112, MP/SP channel divider 154 and mouthpiece support 156, to respective ports 130A and 130B (not shown in FIG. 4) of the secondary channel 130 of the mouthpiece 128.

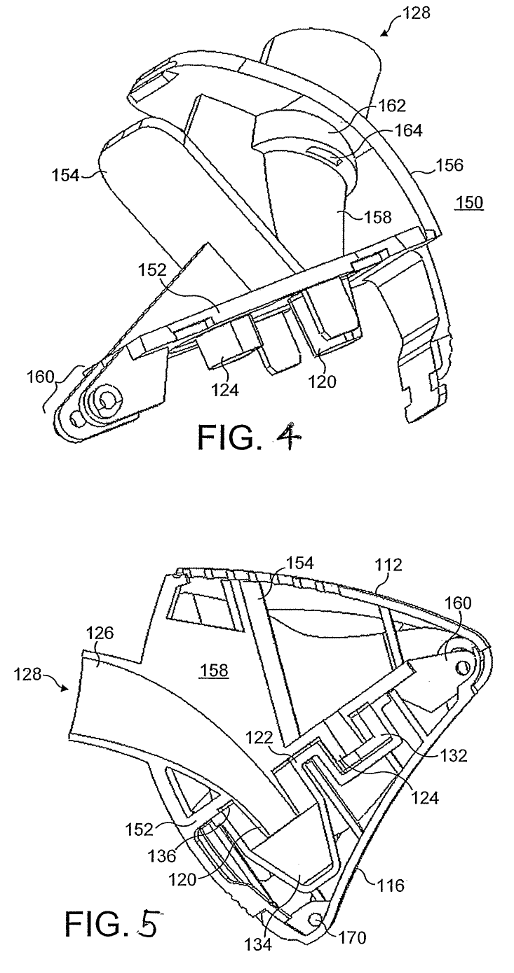

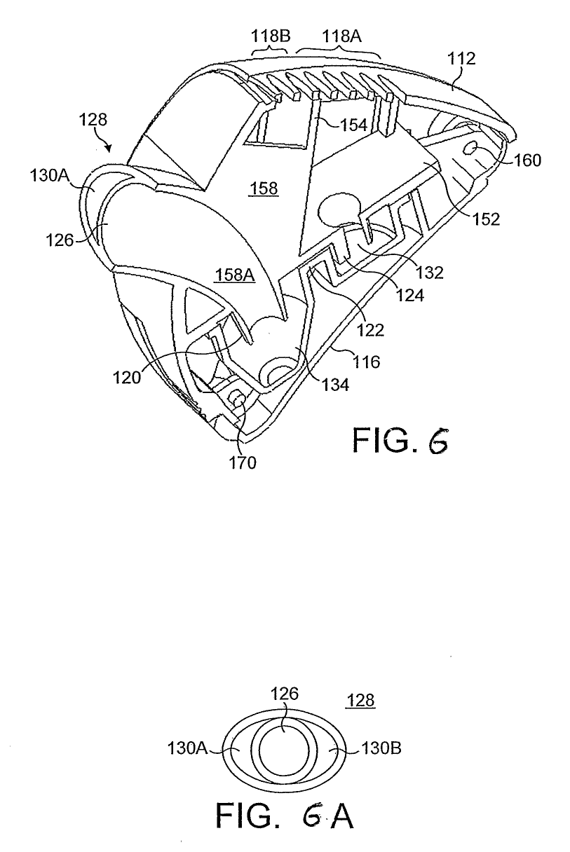

FIGS. 5 and 6 show a sectional view (about a centre plane) and in perspective, (about a centre plane) of the dose holder 116, operatively connected to the pivot assembly 160 of the structure 150. In those Figures, the dose holder 116 is fully rotated toward base portion 152 and supports dose container blister pack 122 with its face member 136 flush against the underside (as shown in FIG. 6) of base portion 152. In this position, the piercing tubes 120 and 124 are shown as pierced through the foil 140 affixed to face member 136. In this position of dose holder 116 and structure 150, the main flow path MP is established, as described below. The drug container blister pack 122 may be removed or replaced by pivoting the dose holder 116 anti-clockwise (as shown in FIG. 5) with respect to the structure 150, to permit clearance for removing and replacing the drug container 122. A pivot assembly 170 (for pivotally supporting the mouthpiece protector 114) is disposed at the left end (as shown in FIGS. 5 and 6) of the dose holder 116. FIG. 6A shows a plan view of the mouthpiece 128, showing channels 126, 130A and 130B.

FIG. 7 shows the inhaler casing 112, drug holder 116, and drug container 122 as shown in FIGS. 5 and 6, and further shows the mouthpiece protector 114 in its open and ready-to-use position, pivoted anti-clockwise (as shown in FIG. 7) relative to the drug holder 116. When the inhaler 110 is not in use, the mouthpiece protector 114 is rotated clockwise to a closed position (as shown in FIG. 18) with respect to drug holder 116. The mouthpiece protector 114 may be resiliently biased towards, or snap-fit into, its closed position; and the drug holder 116 may be resiliently biased toward, or snap-fit into, its closed position, for convenience of a user.

In the use of the inhaler 110, a user carries the inhaler 110 in its closed position (as shown in FIG. 1), with the mouthpiece protector 114 in position over the mouthpiece 128 and the drug holder in its closed position. In order to take a dose of drug, the user pivots the mouthpiece protector 114 to its open position and pivots the drug holder 116 to its open position. The user then inserts a drug container blister pack 122 (with its foil 140 intact) into the drug holder 116. The user then pivots the drug holder 116 to its closed position. This action causes the piercing tubes 120 and 124 to pierce the foil 140 and enter the chambers 134 and 132 respectively. The pivoting of the drug holder 116 to its closed position establishes the main airflow path MP, from ports 118A, through the inlet piercing tube 124, inlet chamber 132, cyclone chamber 134, vortex finder 120 and the cyclone chamber outlet to one channel 126 of the mouthpiece 128. The secondary flow path SP exists at all times from ports 118B to further channels 130A and 130B of the mouthpiece 128, as described above.

The user then places his or her lips about the mouthpiece 128 and inhales through his or her mouth. As a result, the user establishes a primary air flow along the main flow path MP. In the inlet chamber 132, drug and associated carrier particles are entrained into the primary airflow which then passes tangentially into the upper part of the cyclone chamber 134. The resulting reverse-cyclone flow pattern in the cyclone chamber 16 is shown in greater detail in FIG. 8.

The tangentially entering air and cylindrical upper wall portion set up a bulk circulation of air around the periphery of the chamber 134. The inlet to the chamber is angled down slightly so that the air flow is a shallow downward spiral known as a “free” vortex 135a. Due to conservation of angular momentum, the rotational velocity of the free vortex increases as the airflow is constricted by the tapering inner surface of the frusto-conical portion of the chamber 134a. As the free vortex 135a hits the base of the chamber 134b it is reflected to form a tight “forced vortex” 135b inside the free vortex and travelling back up the axis of the chamber.

At the top of the chamber 134 the downwardly projecting end of the outlet tube 120 forms a vortex finder. The vortex finder 120 effectively defines a maximum cut-off circulation radius for entrained particles to exit the chamber. Particles circulating at a radius greater than that of the vortex finder 120 will not escape but will either fall back into the cyclone or fall to the base of the chamber.

As entrained powder particles enter the cyclone chamber 134 to be carried downwardly they circulate around the chamber 134 several times. As they travel, the particles experience a shear force arising the from the relatively high spatial velocity gradients that occur when measured across the two vortices 135a, 135b. This shear force tends to de-aggregate and de-agglomerate the particles so that the average size of the particles is reduced and drug particles circulate separately from carrier particles.

At the base of the chamber the reversal of direction causes the heavier particles, such as the carrier particles, to come out of the main flow to be trapped in eddy currents at the bottom of the chamber or simply to sit at the bottom of the chamber.

The lighter particles which remain entrained travel back up the chamber 134 in the forced vortex 135b giving a further opportunity for de-aggregation. The diameter of the carrier particles is greater than the depth of the boundary layer at the wall of the cyclone chamber and therefore large particles do not remain stationary on the cyclone chamber wall but continue to circulate releasing fine particles throughout the inhalation. It will be seen therefore that in contrast to particles being drawn once round a swirl chamber as is known from the prior art, the flow path obtained in accordance with the invention gives a long path through the chamber and so a long residence time which enhances the de-aggregation efficiency, by increasing the number of opportunities the fine particles have to be removed from the carrier particles. The smaller particles with lower momentum circulate at relatively short radii whereas larger particles with greater momentum circulate at larger radii. At the top of the chamber the vortex finder 120 selects the smaller particles, e.g. those of diameter 5 μm or less, with the rest remaining in the chamber as explained above.

The forced vortex air flow exits the cyclone chamber 134 through the vortex finder 120 and passes along the mouthpiece channel 126 into the mouth of the user. The diverging mouthpiece 128 slows the air flow before it enters the mouth of the user so that it does not impinge forcefully against the back of the user's throat.

FIGS. 9 to 11 show a modified piercer/mouthpiece structure 50 in accordance with the present invention. This structure 50 replaces the structure 150 described in relation to FIG. 4 above. In this embodiment of the invention the cyclone chamber outlet tube 20 is connected to the mouthpiece 28 by means of a plenum portion 58. This plenum portion receives the cyclone chamber outlet vertically (as viewed from FIG. 9) and opens out into the right-hand channel 26 (as viewed from FIGS. 9 to 11) of the mouthpiece. The central axis of the mouthpiece channel 26 is offset by a distance from the central axis A_{c} of the cyclone chamber outlet 20 as is shown most clearly in the schematic representation in FIG. 12. The left-hand channel 30 of the mouthpiece is connected to the bypass air port as in the previously described arrangement.

Returning to FIGS. 9 to 11 it will be seen that rather than an abrupt corner between the cyclone chamber outlet and the mouthpiece, there is a smooth, scroll section 58a which helps to prevent turbulent flow which would increase the risk of powder deposition, contrary to the desired effect.

Operation of the inhaler incorporating the structure shown in FIGS. 9 to 11 is mostly the same as that described with reference to FIGS. 1 to 7. The difference is that as the tightly helically circulating air passes through the cyclone chamber outlet/vortex finder 20, it encounters the plenum portion 58. The shape of this plenum portion 58 and in particular the fact that the mouthpiece channel 26 is offset from the centre of the circulating flow means that air can continue out along the mouthpiece channel 26 at a tangent to the circulation which minimises the tendency for entrained powder particles to be deposited on the inner surface of the mouthpiece and minimises the flow resistance presented by the inhaler overall. The smooth scroll portion 58a further enhances this since it follows the path naturally followed by the air.

The efficacy of the preferred reverse-flow cyclone arrangement will now be demonstrated using the following examples.

FIG. 13 shows five different cyclone chamber configurations A-E used in a performance test. The cyclone chamber diameters range from 10 to 20 mm. FIG. 14 shows the performance test results for the cyclones A-E compared to two conventional dry powder inhalers. The fine particle fraction achieved using the cyclones A-E is seen to be over 69%, and as much as 81%, compared to only 30-40% for conventional dry powder inhalers. This results from the deposition of large particles above the cut-off size in the base of the cyclone chamber, so that the fine particle fraction is greatly enhanced. The size of the particles separated by the cyclones A-E was also reduced to 2-3 μm in all configurations. Thus cyclone chambers of these configurations separate out particles of a much finer, respirable size than can be achieved by conventional dry powder inhalers, therefore concentration of fine particles in the emitted dose is increased compared to the conventional formulation.

It will be appreciated by those skilled in the art that the embodiment set out above gives a simple and convenient arrangement for a dry powder inhalers in which particles which are too large are retained in the device thus raising the Fine Particle

Fraction of what is inhaled and reducing the problems arising with inhaling particles which are too large. At the same time it reduces deposition of active drug on the inside of the inhaler which can be dislodged to the detriment of dose content uniformity. This provides significant benefit to the user in a commercially attractive package.

Some key features of the preferred embodiment include the following. Firstly, a reverse-flow cyclone to efficiently de-aggregate the respirable (fine) drug particles from coarse carrier fraction (e.g. lactose). This is achieved by increasing the residence time of the particles (therefore a greater number of opportunities for separation), and by maximizing the shear forces for a given energy input. Secondly, the reverse-flow cyclone separates and retains the coarse carrier fraction—i.e. only respirable (fine) drug particles are emitted upon inhalation. Thirdly the use of bypass airflow to control the separation efficiency of the reverse-flow cyclone, and to tailor the airflow resistance of the device.

Fourthly the cyclone geometry being on a disposable component, to maximize dose content uniformity arising from this part of the inhaler, by preventing carry-over of drug particles between doses.

Fifthly, formulation is pre-metered into moisture-proof blister, therefore accurate dose mass, and performance independent of environmental conditions. Finally, a disposable blister which retains the non respirable fraction aerodynamically during inhalation and mechanically after inhalation.

However the described embodiment is only examples of the large number of possible implementations of the invention and many variants and alternatives are possible. For example it is not essential for the cyclone chamber to be provided on a disposable part; the inhaler could be made as an integral unit whilst still retaining many of the benefits of the invention, particularly those pertaining to the provision of a reverse-cyclone with a bypass airflow path.

In alternative embodiments, there is only the primary airflow path, without any secondary (bypass) airflow path.

A blister pack embodying a further invention is now described with reference to FIGS. 15 to 17. The blister pack 200 comprises a moulded plastics base part 202 which provides two interlinked chambers 204, 206. The rightmost chamber 204 (from the point of view of FIGS. 15 to 17) is a cylindrical swirl chamber. The leftmost chamber 206 is a frusto-conical chamber. The two chambers 204, 206 are linked by a passageway (not visible) at their uppermost ends. The blister pack is therefore similar in many respects to that described with reference to FIG. 2 and 3.

However rather than simply being sealed by a foil membrane, the base part 202 is closed by a grommet cover 208 and a protective layer 210 (see FIG. 15). The grommet cover 208 is attached over the top of the base part 202, e.g. by ultrasonic welding. As may be seen in FIGS. 16 and 17, two apertures 212, 214 are defined in the grommet cover 208, one above each of the chambers 204, 206. The apertures are D-shaped and circular respectively.

The grommet cover 208 is preferably made of an elastically compliant material—e.g. silicone rubber. In the embodiment shown in FIGS. 15 to 17 the grommet cover is a simple single material lamina. However it is equally envisaged that the grommet cover could be of composite construction—e.g. with a suitably compliant material provided around the aperture(s) attached e.g. by co-moulding, to a different material forming the rest of the cover. Similarly the grommet cover need not extend fully across an open face of the base part; for example the base part could extend some way over the top of the chambers.

The protective layer 210 is attached to the grommet cover 208 with a non-permanent adhesive so as to cover the apertures 212, 214. A small tab 216 is left free of adhesive to allow the protective layer 210 to be ripped off.

To use the blister pack 200 first the protective layer is peeled off and the pack is then installed in an inhaler such as the one described in relation to the earlier invention. As the inhaler is closed again after installing the pack, the two ‘piercing tubes’ 120, 124 will pass through their respective apertures 214, 212. The apertures 212,214 are chosen to be slightly smaller in size than the size of the respective tubes 124, 120 so that as the tubes pass through the apertures the compliance of the grommet cover 208 enables an air-tight seal to be formed around each tube. The inhaler then operates as previously described with the air-tight seals around the tubes 120, 124 ensuring that efficient airflows, and in particular an efficient reverse-cyclone flow is set up during use. As there is no foil membrane there is no risk of flaps of foil hindering the desired air flows.

1. A dry powder inhaler comprising: a cyclone chamber having a cyclone chamber outlet extending substantially axially there from; a mouthpiece channel having a central axis; and a plenum portion connecting said cyclone chamber outlet and said mouthpiece channel, the mouthpiece channel axis being offset from, and making a non-parallel angle with, the axis of the cyclone outlet such that in use air exits the cyclone outlet at least partly tangentially into the mouthpiece.

2. An inhaler as claimed in claim 1 wherein the cyclone chamber is so shaped that at least a part of the chamber decreases in cross-sectional area in a direction away from its air inlet, so as thereby in use to set up a reverse flow cyclone in the chamber.

3. An inhaler as claimed in claim 1 wherein the cyclone chamber is provided as an integral part of the inhaler.

4. An inhaler as claimed in claim 1 wherein the cyclone chamber is provided fully or partially by a removable part.

5. An inhaler as claimed in claim 4 wherein one or more doses of the powdered substance is also provided on the removable part.

6. An inhaler as claimed in claim 5 wherein said doses are individually sealed.

7. An inhaler as claimed in claim 1 wherein the chamber is protected by a frangible membrane.

8. An inhaler as claimed in claim 7 wherein said frangible membrane is pierced upon installation into the inhaler.

9. An inhaler as claimed in claim 1 wherein said chamber comprises an air inlet arranged to direct air in such a way that it circulates around the periphery of the chamber.

10. An inhaler as claimed in claim 1 wherein comprising a medicament powder is stored within the cyclone chamber.

11. An inhaler as claimed in claim 1 wherein the diameter of the cyclone chamber is between 5 and 100 mm, more preferably between 5 and 50 mm and most preferably between 8 and 20 mm.

12. An inhaler as claimed in claim 1 wherein comprising a main airflow path which passes through the cyclone chamber and a bypass airflow path bypassing the cyclone chamber; wherein the main and bypass airflow paths communicate with the mouthpiece.

13. An inhaler as claimed in claim 12 comprising means for varying the flow resistance of the bypass air flow path such that said resistance is decreased at increasing inhalation flow rates.

14. An inhaler as claimed in claim 12 wherein said mouthpiece is divided, with one channel for the main airflow and the other channel for the bypass airflow, such that the main and bypass airflows do not meet at all inside the inhaler.

15. A mouthpiece arrangement for a dry powder inhaler comprising a cyclone chamber outlet for connection in use with a cyclone chamber; a mouthpiece channel having a central axis; and a plenum portion connecting said cyclone chamber outlet and said mouthpiece channel, the mouthpiece channel axis being offset from, and making a non-parallel angle with, the axis of the cyclone chamber outlet.

16. A dry powder inhaler as claimed in claim 1 wherein the axis of the mouthpiece channel extends at an obtuse angle from the cyclone outlet axis.

17. A blister pack for use with a dry powder inhaler and including a dose of powder for inhalation, the blister comprising: a base part comprising at least one chamber; a grommet cover extending partly over said chamber and defining an aperture above the chamber; and a protective layer covering at least said aperture, wherein the edge of the grommet cover defining the aperture is compliant so as in use to form a seal around a tube passing through the aperture.

18. A blister pack as claimed in claim 17 wherein the edge of the grommet cover defining the aperture is elastically compliant.

19. A blister pack as claimed in claim 17 wherein said protective layer or a portion thereof is removable.

20. A blister pack as claimed in claim 17 wherein said chamber is so shaped that at least a part of the chamber decreases in cross-sectional area in a direction away from its air inlet, so as thereby in use to set up a reverse flow cyclone in the chamber.

21. A blister pack as claimed in claim 17 comprising a second aperture defined in a grommet cover with a compliant edge.

22. A dry powder inhaler including a blister pack as claimed in claim 17.

23. An inhaler as claimed in claim 22 comprising a tube which can be introduced into the chamber through the aperture in the grommet cover, the tube being slightly larger than the aperture so that the grommet cover forms a seal around the tube.

24. A dry powder inhaler comprising a dose of powder for inhalation, a body part defining at least one chamber; a grommet cover extending partly over said chamber and defining an aperture above the chamber; and a protective layer covering at least said aperture, wherein the edge of the grommet cover defining the aperture is compliant so as in use to form a seal around a tube passing through the aperture.

25. An inhaler as claimed in claim 24 further comprising a tube which can be introduced into the chamber through the aperture in the grommet cover, the tube being slightly larger than the aperture so that the grommet cover forms a seal around the tube.

26. An inhaler as claimed in claim 24 wherein said protective layer or a portion thereof is removable.

27. An inhaler as claimed in claim 24 wherein said chamber is so shaped that at least a part of the chamber decreases in cross-sectional area in a direction away from its air inlet, so as thereby in use to set up a reverse flow cyclone in the chamber.

28. An inhaler as claimed in claim 24 comprising a second aperture defined in a grommet cover with a compliant edge.

29. A mouthpiece arrangement as claimed in claim 15 wherein the axis of the mouthpiece channel extends at an obtuse angle from the cyclone outlet axis.
